(19) United States
(12) Patent Application Publication
(10) Pub. No.: US 2014/0099237 A1
(43) Pub. Date: Apr. 10, 2014

(54) BIOMEDICAL MONITORING SYSTEM COMBINING A MOBILE DEVICE

(71) Applicant: ALCOR MICRO CORP., Taipei City (TW)

(72) Inventors: Chi Tung CHANG, Taipei City (TW); Shih Min LAN, Taipei City (TW); Hao Yu CHENG, Taipei City (TW)

(73) Assignee: ALCOR MICRO CORP., Taipei City (TW)

(21) Appl. No.: 14/040,928

(22) Filed: Sep. 30, 2013

(30) Foreign Application Priority Data
Oct. 1, 2012 (TW) 101136283

(51) Int. Cl. A61B 5/00; G01N 33/50

(52) U.S. Cl. CPC A61B 5/6898; G01N 33/50
USPC 422/82.01; 600/364

(57) ABSTRACT

The present invention provides a biomedical monitoring system combining a mobile device. Signals can be transmitted between the sensing device and the mobile device via the audio interface by modulating and demodulating the audio signals or encoding and decoding the digital signals. Thereby, it can be applied extensively to mobile devices having audio jacks and thus bringing more convenience. In addition, because the mobile devices inherently have the functions of operational processes, transmission, storage, interface operations, result displaying, network connection, image extraction, and power supply, the required components in sensing devices can be simplified substantially and hence reducing the volume and the manufacturing cost.

### FIELD OF THE INVENTION

The present invention relates generally to a biomedical monitoring system, and particularly to a biomedical monitoring system combining a mobile device, which uses the functions of operational processes, transmission, storage, interface operations, result displaying, network connection, image extraction, and power supply provided by the mobile device for reducing the required components in a sensing device.

### BACKGROUND OF THE INVENTION

In recent years, the biomedical sensing devices and the biomedical test strips for personal or home applications outside medical institutions are developed continually. Thereby, general people or patients can measure and monitor their own biomedical parameters at home or anywhere and thus achieving the purpose of homecare or health care. For facilitating measurement of biomedical parameters at home or anywhere, this kind of biomedical sensing devices are mostly designed compact in the appearance for convenient carrying or storage. Nonetheless, due to the limitation of their volume, the displaying area of the devices is insufficient, which is tiring for the elderly or users having poor eyesight in reading the measurement result, let alone in displaying a plurality of measurement results for presenting the trend of variation in biomedical parameters.

For achieving the purpose of homecare or health care, long-term measurement results of biomedical parameters are more important than a single measurement result. Most of current biomedical sensing devices have a data transmission interface for users to transmit measurement results to other electronic devices for storage and recording, and relieving the inconvenience of handwriting or inputting to other electronic devices in early days. The specification of the data transmission interface in current biomedical sensing devices for homecare is mostly the USB interface, which is convenient for connecting to computer devices and transmitting data. Nonetheless, compared with biomedical sensing devices, the computer devices always have larger volume, diminishing the original target of ease of carrying by shrinking biomedical sensing devices. Thereby, combining biomedical sensing devices with mobile device having similar compact size, such as the popular smartphones, should be a better choice than combining with computer devices. In addition, after combining with mobile devices, not only their storage space can be used, their displaying function can also be further exploited and thus removing the limitation of screen displaying as described above.

While combining biomedical sensing devices with smartphones, the problem of differences in specifications of transmission interface and the in file formats in commercial smartphones occurs. In addition to letting users select different transmission wires or conversion wires according to smartphones for complying with the required specifications of transmission interface, the manufacturers of biomedical sensing devices also need to have certifications and protocol with the suppliers of various smartphones so that the smartphones can support their file formats. Otherwise, the biomedical sensing devices will not be compatible with the smartphones.

Accordingly, the inventors of the present invention conceive and develop sensing devices capable of transmitting data with arbitrary mobile devices. The inventors select the audio jack (the earphone jack), which is most popular in all mobile devices, and the audio interface as the data transmission interface between the sensing devices and mobile devices and hence can be applicable extensively to various mobile devices. Besides, when the mobile devices convert the electrical data generated by the sensing device by measuring biomedical parameters to biomedical data, the biomedical data files, which are accessible by the mobile devices and the statistics of the biomedical data files can be gathered by the mobile devices, can be generated according to the supported file data formats. Thereby, the convenience of using the biomedical monitoring system can be enhanced significantly.

### SUMMARY

An objective of the present invention is to provide a biomedical monitoring system combining a mobile device, which selects the audio interface as the transmission interface for being applied extensively to mobile devices having audio jacks.

An objective of the present invention is to provide a biomedical monitoring system combining a mobile device, which uses the functions of operational processes, transmission, storage, interface operations, result displaying, network connection, image extraction, and power supply provided by mobile devices for reducing substantially the required components in sensing devices, the size, and the manufacturing costs.

An objective of the present invention is to provide a biomedical monitoring system combining a mobile device, which uses the function of operational processes in mobile devices. By using algorithms, the electrical data generated by the measurement of sensing devices are converted to biomedical data and written to biomedical data files in formats supported by the mobile devices.

An objective of the present invention is to provide a biomedical monitoring system combining a mobile device, which uses the gravity sensing unit or gyroscope unit in the mobile devices to sense the movement or rotation of the mobile devices by users, so that the mobile devices can execute the functions corresponding to the movement or rotation.

An objective of the present invention is to provide a biomedical monitoring system combining a mobile device, which uses the image extraction unit contained in the mobile devices to extract images, so that the mobile devices can execute the functions corresponding to the images.

An objective of the present invention is to provide a biomedical monitoring system combining a mobile device, which enables the sensing unit of the sensing device to read a plurality of types of biomedical strips and thus providing a compound measurement function of biomedical parameters.

For achieving the objectives described above, the present invention provides a biomedical monitoring system combining a mobile device, which comprises a sensing device and a mobile device. The sensing device comprises a sensing unit and a first audio interface. The sensing unit is connected electrically to the first audio interface. The first audio interface is further connected with an audio plug. The mobile device comprises a processing unit and a second audio interface. The processing unit is connected electrically to the second audio interface. The second audio interface is further connected with an audio jack. In addition, the audio plug is coupled with the audio jack. Thereby, the first audio interface of the sensing device is connected electrically with the second audio interface of the mobile device. While using, the sensing device measures at least a biomedical parameter. Then at least an electrical datum generated by measuring the biomedical parameter is transmitted to the processing unit via the first audio interface, the audio plug, the audio jack, and the second audio interface sequentially. After the processing unit receives the electrical datum, it converts the electrical datum to at least a biomedical datum.

The circuits of the first and second audio interfaces can be divided into a left sound channel and a right sound channel. The left sound channel of the first audio interface is connected electrically with the left sound channel of the second audio interface; the right sound channel of the first audio interface is connected electrically with the right sound channel of the second audio interface. When the electrical datum is transmitted using the left sound channel, the right sound channel can be used for supplying power from the mobile device to the sensing device. When the electrical datum is transmitted using the right sound channel, the left sound channel can be used for supplying power from the mobile device to the sensing device.

The sensing device further comprises a microcontroller unit, which is connected electrically to the sensing unit and the first audio interface. Besides, it can be combined with the functions of a modulation unit or an encoding unit for controlling the transmission of the electrical datum. The modulation unit can modulate the electrical datum and generate an audio signal; the encoding unit can encode the electrical datum and generate a digital signal. Then the audio signal or the digital signal is transmitted to the processing unit via the first audio interface, the audio plug, the audio jack, and the second audio interface. After the processing unit receives the audio signal or the digital signal, a demodulation unit or a decoding unit is used for demodulating or decoding the audio signal or the digital signal and recovering the audio signal or the digital signal to the electrical datum, which is further converted to the biomedical datum. Because most functions are executed by the mobile device except measuring the biomedical parameter, the sensing device can only have the functions of measuring the biomedical parameter, generating the electrical datum, and controlling the transmission of the electrical datum, significantly simplifying the components of the sensing device. Because the sensing device also needs to receive and interpret the messages or data transmitted by the mobile device, the mobile device has to include a modulation unit or an encoding unit; the sensing device has to include a demodulation unit or a decoding unit. Thereby, the mobile device and the sensing device can interact with each other.

The mobile device can further comprise a memory unit, a displaying unit, an operational unit, an image extraction unit, a voice unit, or a terminal unit connected electrically to the processing unit.

The memory unit can store the biomedical datum (or the plurality of the biomedical data) and at least an application program. The application program includes at least an algorithm. The processing unit converts the electrical datum to the biomedical datum through the algorithm. The application program can be developed according to different mobile devices so that biomedical datum can be written to a biomedical data file, which is in the file format supported by the mobile device and further stored. The application program can also include other functions such as initializing the software, hardware, and firmware of the mobile device and the sensing device. Hence, the sensing device can execute the functions of measuring the biomedical parameter, compiling statistics and analysis of a plurality of biomedical data, presenting the results of statistics and analysis in graphics, retrieving previous records of biomedical data, and calibrating the electrical datum or the biomedical datum. In addition, the application program can execute automatically when the mobile device detects the sensing device for facilitating the operating process.

The displaying unit can display the graphics and tables of at least a biomedical datum or a plurality of biomedical data generated by the statistics of the processing unit. It can also display a graphic user interface, Users can use the graphic user interface together with the operating unit for commanding the biomedical monitoring system to measure the biomedical parameter, display the biomedical data or the graphics and tables generated b the plurality of biomedical data, and calibrate the electrical datum or the biomedical datum.

The operating unit can comprise a touch unit, a button unit, a gravity sensing unit, and gyroscope unit. The touch unit can generate a touch signal corresponding to the touch action of users; the button unit can generate a button signal corresponding to the button action of users; the gravity sensing unit can generate a movement signal corresponding to the movement of the mobile device by users; and the gyroscope unit can generate a rotation signal corresponding to the rotation of the mobile device by users. The touch signal, the button signal, the movement signal, and the rotation signal are named an operating signal. The operating unit can transmit the operating signal to the processing unit so that the processing unit can execute the functions corresponding to the operating signal. For example, the movement signal or the rotation signal enables the mobile device to initialize of the sensing device before measuring and enables the sensing device to start measuring the biomedical datum. The rotation signal can alter the biomedical datum displayed on the displaying unit and presenting the effect of page pulling.

The image extraction unit can extract an image and transmit the image to the processing unit for enabling the processing unit to execute the function corresponding to the image. The image can be generated by extracting a barcode of the biomedical test strip by the image extraction unit. The barcode can include the calibration condition of the biomedical test strip. Because the concentration values of the biological reagent attached to the test regions of the biomedical test strips in different manufacturing batches or in different manufacturing blocks in the same batch are not necessarily identical or uniform, manufacturers will sample the test strips of each batch or each manufacturing block, measure the concentration values of the biological reagent attached to the test regions of the biomedical test strips, and calculate the calibration conditions for reducing the influence of the variations in the concentration of the biological reagent during manufacturing. After the processing unit receives the image, it can decode the barcode contained in the image. Then the processing unit acquires the calibration condition according to the barcode for calibrating the biomedical datum.

The voice unit can output the biomedical datum in voice, so that users can know the measurement result by hearing. The terminal unit can be connected to the Internet. It can download an updated firmware or an updated application program from the Internet for updating the firmware in the sensing device or the application program in the mobile device. In addition, as described above, after acquiring the image, the calibration condition can be given by connecting to the Internet according to the decoded barcode. Alternatively, the image can be uploaded directly. The internet will decode the barcode and give the calibration condition.

### BRIEF DESCRIPTION OF THE DRAWINGS

FIG. 1 shows a schematic diagram of the connection of devices according to a first embodiment of the present invention; and

FIG. 2 shows a schematic diagram of the connection of devices according to a second embodiment of the present invention.

### DETAILED DESCRIPTION

In order to make the structure and characteristics as well as the effectiveness of the present invention to be further understood and recognized, the detailed description of the present invention is provided as follows along with embodiments and accompanying figures.

The biomedical monitoring system combining the mobile device according the present invention has the following features. It selects the audio interface as the transmission interface for being applied extensively to mobile devices having audio jacks. It uses the functions of operational processes, transmission, storage, interface operations, result displaying, network connection, image extraction, and power supply provided by mobile devices for reducing substantially the required components in sensing devices, the size, and the manufacturing costs. It uses the function of operational processes in mobile devices. By using algorithms, the electrical data generated by the measurement of sensing devices are converted to biomedical data and written to biomedical data files in formats supported by the mobile devices. It uses the gravity sensing unit or gyroscope unit in the mobile devices to sense the movement or rotation of the mobile devices by users, so that the mobile devices can execute the functions corresponding to the movement or rotation. It uses the image extraction unit contained in the mobile devices to extract images, so that the mobile devices can execute the functions corresponding to the images. It enables the sensing unit of the sensing device to read a plurality of types of biomedical strips and thus providing a compound measurement function of biomedical parameters.

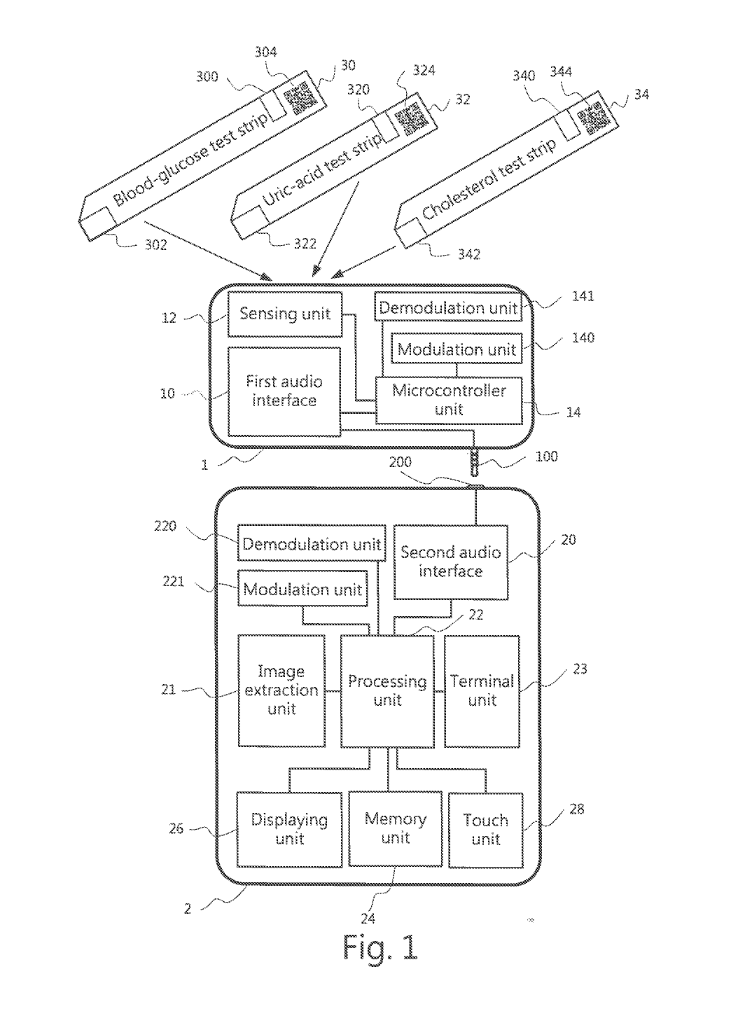

Please first refer to FIG. 1, which shows a schematic diagram of the connection of devices according to a first embodiment of the present invention. As shown in the figure, the biomedical monitoring system combining the mobile device according to the present invention comprises a sensing device 1 and a mobile device 2. The sensing device 1 comprises a first audio interface 10, a sensing unit 12, a microcontroller unit 14, a modulation unit 140, and a demodulation unit 141. The sensing unit 12 is connected electrically with the first audio interface 10; the microcontroller unit 14 is connected electrically with the modulation unit 140, the demodulation unit 141, the sensing unit 12, and the first audio interface 10, respectively. Besides, the first audio interface 10 is connected electrically with an audio plug 100. The mobile device 2 comprises a second audio interface 20, a processing unit 22, a demodulation unit 220, a modulation unit 221, a memory unit 24, a displaying unit 26, a touch unit 28, an image extraction unit 21, and a terminal unit 23. An audio jack 200 is connected electrically with the second audio interface 20. The audio plug 100 can be plugged into the audio jack 200 for connecting electrically the sensing device 1 and the mobile device 2. The second audio interface 20 is connected electrically with the processing unit 22. The processing unit 22 is further connected with the demodulation unit 220, the modulation unit 221, the memory unit 24, the displaying unit 26, the touch unit 28, the image extraction unit 21, and the terminal unit 23, respectively.

According to the present embodiment, the sensing device 1 is a compound reading device for test strips capable of measuring three biomedical parameters. It can read a blood-glucose test strip 30, a uric-acid test strip 32, or a cholesterol test strip 34. These test strips all include an electrode region 300, 320, or 340, a test region 302, 322, or 342, and a barcode region 304, 324, or 344.

Because the concentration values of the biological reagent attached to the test regions of the biomedical test strips in different manufacturing batches or in different manufacturing blocks in the same batch are not necessarily identical or uniform, manufacturers will sample the test strips of each batch or each manufacturing block, measure the concentration values of the biological reagent attached to the test regions of the biomedical test strips, and calculate a calibration condition for reducing the influence of the concentration of the biological reagent on measurement. Before using, the image extraction unit 21 scans the barcode region 304, 324, or 344 and produces an image. The terminal unit 23 is connected to the Internet. The calibration condition provided by the manufacturer of the blood-glucose test strip 30, the uric-acid test strip 32, or the cholesterol test strip 34 is given according to a barcode generated by decoding the image for calibrating the subsequently measured electrical datum, the biomedical datum converted by the electrical datum, or the algorithm (parameter) for converting the electrical datum.

While using, smear the whole blood drop gathered from the blood capillary of a human body on the test region 302, 322, or 342 of the blood-glucose test strip 30, the uric-acid test strip 32, or the cholesterol test strip 34 to let the whole blood from blood capillary react with the biological reagent on the test region 302, 322, or 342. The biological reagent on the test region 302 of the blood-glucose test strip 30 can react with the glucose in the whole blood from blood capillary and generate electrons accumulating on the electrode region 300. The biological reagent on the test region 3202 of the uric-acid test strip 32 can react with the uric acid in the whole blood from blood capillary and generate electrons accumulating on the electrode region 320. The biological reagent on the test region 342 of the cholesterol test strip 34 can react with the total cholesterol in the whole blood from blood capillary and generate electrons accumulating on the electrode region 340. Afterwards, when the blood-glucose test strip 30, the uric-acid test strip 32, or the cholesterol test strip 34 is inserted into the sensing device 1, the sensing unit 12 can give an electrical datum according to the number of electrons on the electrode region 300, 320, or 340. The electrical datum is a voltage value with the unit of microvolt. The microcontroller unit 14 modulates the electrical datum using the modulation unit 140 and generates an audio signal. In addition, the microcontroller unit 14 controls the audio signal to be transmitted to the processing unit 22 via the first audio interface 10, the audio plug 100, the audio jack 200, and the second audio interface 20.

After the processing unit 22 receives the audio signal, it uses the demodulation unit 220 to demodulate and recover the audio signal to the electrical datum. Besides, an algorithm contained in an application program stored in the memory unit 24 converts the electrical datum to a biomedical datum and displays the biomedical datum on the displaying unit 26. The biomedical datum is a blood glucose value, a uric acid value, or a total cholesterol value with the unit of mg/100 mL or mmol/L. While converting, the processing unit 22 will match the calibration condition for calibrating the electrical datum, the biomedical datum, or the parameter of the algorithm and giving a more accurate measurement result.

The processing unit 22 can edit a biomedical data file of the biomedical datum in the file format supported by the mobile device 2 and store the biomedical data file to the memory unit 24. By means of a graphic user interface displayed on the displaying unit 26 and the touch unit 28, users can operate the mobile device 2 and enable an operating unit t generate an operating signal corresponding to the operation of the users. The processing unit 22 then responds to the operating signal (according to the present embodiment, the operating unit is the touch unit 28; the operating signal is a touch signal). By using the application program (and the firmware), the processing unit 22 executes the functions of starting the sensing device 1, compiling statistics and analysis of a plurality of biomedical data, presenting the graphics and tables of the biomedical data, retrieving previous records of biomedical data, and performing calibration. While starting the sensing device 1, it is required to transmit signals from the mobile device 2 to the sensing device 1. Thereby, the modulation unit 221 in the mobile device 2 can be used for modulating the signals to audio signals. After the audio signals are transmitted to the sensing device 1, the demodulation unit 141 is used for demodulation. Moreover, after the terminal unit 23 is connected to the network, the version of the application program can be check as well and an updated application program can be downloaded.

In addition to the touch unit 28 used in the present embodiment, some mobile devices have a button unit. Users can use the button unit to generate the operating signal (button signal) for submitting instructions to the biomedical monitoring system combining the mobile device. Besides, according to the present embodiment, the modulation units 140, 221 and the demodulation units 141, 220 are used for modulating and demodulating data signals for transmission. Alternatively, an encoding unit and a decoding unit can be used instead for encoding and decoding data signals. Then the data signals can be transmitted in the form of a digital signal, as shown in the second embodiment. Furthermore, the sensing device 1 can also acquire the electrical datum by measuring resistance and current in the electrode regions 300, 320, 340. The barcode regions 304, 324, 344 can also be disposed on the package of the blood-glucose test strip 30, the uric-acid test strip 32, or the cholesterol test strip 34. Alternatively, a separate barcode paper can be attached. By omitting printing on each strip, the overall cost of strips can be further reduced.

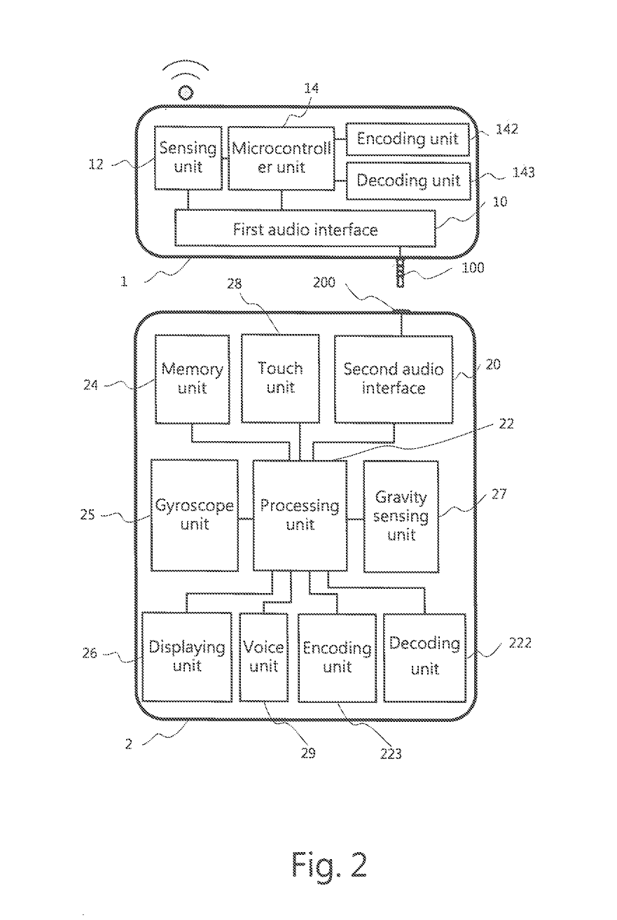

Please refer to FIG. 2, which shows a schematic diagram of the connection of devices according to a second embodiment of the present invention. As shown in the figure, the biomedical monitoring system combining the mobile device according to the present invention is a body-temperature monitoring system, which comprises a sensing device 1 and a mobile device 2. The sensing device 1 comprises a first audio interface 10, a sensing unit 12, a microcontroller unit 14, an encoding unit 142, and a decoding unit 143. The sensing unit 12 is connected electrically with the first audio interface 10; the microcontroller unit 14 is connected electrically with the encoding unit 142, the decoding unit 143, the sensing unit 12, and the first audio interface 10, respectively. Besides, the first audio interface 10 is connected electrically with an audio plug 100. The mobile device 2 comprises a second audio interface 20, a processing unit 22, a decoding unit 222, an encoding unit 223, a memory unit 24, a displaying unit 26, a touch unit 28, a gyroscope unit 25, a gravity sensing unit 27, and a voice unit 29. An audio jack 200 is connected electrically with the second audio interface 20. The audio plug 100 can be plugged into the audio jack 200 for connecting electrically the sensing device 1 and the mobile device 2. The second audio interface 20 is connected electrically with the processing unit 22. The processing unit 22 is further connected with the decoding unit 222, the encoding unit 223, the memory unit 24, the displaying unit 26, the touch unit 28, the gyroscope unit 25, the gravity sensing unit 27, and the voice unit 29, respectively.

The sensing device 1 according to the present embodiment is a non-contact infrared temperature sensor. The sensing device 1 can sense the infrared radiated from the body parts (such as the forehead) of a creature and the mobile device 2 can calculate the body temperature of the measured subject. When a user picks the mobile device 2 and prepares to measure the body temperature, the gravity sensing unit 27 detects velocity and displacement of the mobile device 2 and sends a movement signal to the processing unit 22. The processing unit 22 then executes an application program stored in the memory unit 24 for initializing the software and hardware (or firmware) of the sensing device 1 and mobile device 2 prior to measurement. While initializing the sensing device 1, the mobile device 2 needs to send signals to the sensing device 1. Thereby, the encoding unit 223 in the mobile device 2 can be used for encoding the signals to digital signals. After the digital signals are transmitted to the sensing device 1, they will be decoded by the decoding unit 143.

While measuring, the user generates a touch signal via a graphic user interface displayed on the displaying unit 26 and the touch unit 28. The processing unit 22 generates a measurement signal after receiving the touch signal. The measurement signal is transmitted to the microcontroller unit 14 via the second audio interface 20, the audio jack 200, the audio plug 100, and the first audio interface 10. The microcontroller unit 14 instructs the sensing unit 12 to perform measurement. The sensing unit 12 receives the infrared energy radiated from the body surface of a creature and generates an electrical datum, which is just the infrared energy value (converted in millivolt). The microcontroller unit 14 encodes the electrical datum via the encoding unit 142 and generates a digital signal, which is sent to the processing unit 22 via the first audio interface 10, the audio plug 100, the audio jack 200, and the second audio interface 20.

After the processing unit 22 receives the digital signal, the decoding unit 222 decodes and recovers the digital signal to the electrical datum. Then an algorithm contained in an application program stored in the memory unit 24 converts the electrical datum to a biomedical datum, which is further displayed on the displaying unit 26 or read by the voice unit 29. The biomedical datum is just the body temperature of the measured subject in the unit of degrees Celsius or degrees Fahrenheit.

The processing unit 22 can edit the biomedical datum to a biomedical data file with the file format supported by the mobile device 2 and store the biomedical data file to the memory unit 24. The user can query the past biomedical data file and enable the processing unit 22, together with the application program, to present a plurality of biomedical data generated by multiple times of measurement in graphics and tables for facilitating the user to perform long-term observation and monitoring of body temperature. While querying the biomedical data, the user can alter the tilt angle of the mobile device 2 for achieving the effect of page pulling (similar to a mouse wheel). The gyroscope unit 25 senses the variation of the angle of the mobile device 2 and generates a rotation signal, which is transmitted to the processing unit 22. The processing unit 22 enables the display unit 26 to change the displayed biomedical data according to the rotation signal for producing the effect of widow pulling. The rotation signal, the touch signal described above, and the movement signal are all operating signals. The processing unit 22, together with the application program, can execute the functions corresponding to the operating signals.

Similar to the first embodiment, according to the present embodiment, a button unit can be used for replacing the touch unit 28. Users can use the button unit to generate the operating signal (the button signal) for instructing the biomedical monitoring system combining mobile device. In addition, the encoding units 142, 223 and the decoding units 143, 222 used in the present embodiment can also be replaced by a modulation unit and a demodulation unit described in the first embodiment for transmitting signals or data via an audio signal.

Furthermore, the sensing device 1 can be a non-contact blood glucose meter. The electrical signal is generated by contacting the body surface of a user and measuring an oxygen saturation of a limb. Then, similar to the technology disclosed in the first or second embodiment described above, use the mobile device 2 to process the electrical signal and transmit signals bidirectionally with the sensing device 1 for performing the functions of calculating, displaying, operating, or power supplying and thus achieving the purpose of biomedical monitoring.

To sum up, the present invention provides a biomedical monitoring system combining a mobile device. The calculating unit, the storage unit, the displaying unit, the battery or the power cord, the operating unit originally disposed in the sensing device are replaced by the functions, including operational processes, data storage, result displaying, power supplying, and interface operation, provided by the mobile device. Thereby, the required components, the size, and the manufacturing cost of the sensing device can be lowered significantly. Moreover, the functions of image extraction, network connection, movement and rotation sensing provided by the mobile device can be used as well for facilitating measurement calibration, software and hardware updating, and dynamic operations and making the biomedical monitoring system more complete and convenient. Besides, according to the present invention, the audio interface is selected as the interface for transmission. Thereby, it can be applied to various mobile devices having audio jacks and thus making its applications more extensive.

Accordingly, the present invention conforms to the legal requirements owing to its novelty, nonobviousness, and utility. However, the foregoing description is only embodiments of the present invention, not used to limit the scope and range of the present invention. Those equivalent changes or modifications made according to the shape, structure, feature, or spirit described in the claims of the present invention are included in the appended claims of the present invention.

1. A biomedical monitoring system combining a mobile device, comprising:
a sensing device, comprising a sensing with and a first audio interface, said first audio interface connected electrically with said sensing unit and an audio plug, respectively, and said sensing device measuring at least a biomedical parameter, generating an electrical datum, and transmitting said electrical datum outwards via said first audio interface; and
a mobile device, comprising a processing unit and a second audio interface, said second audio interface connected electrically with said processing unit and an audio jack, respectively, said audio plug plugging into said audio jack, said processing unit receiving said electrical datum via said second audio interface and converting said electrical datum to at least a biomedical datum.

2. The biomedical monitoring system combining the mobile device of claim 1, wherein said sensing device comprises a microcontroller unit connected electrically with said sensing unit and said first audio interface, respectively.

3. The biomedical monitoring system combining the mobile device of claim 2, wherein said sensing device comprises a modulation unit and said mobile device comprises a demodulation unit; said microcontroller unit uses said modulation unit to modulate said electrical datum an generate an audio signal; and said processing unit receives said audio signal and uses said demodulation unit to demodulate said audio signal and give said electrical datum.

4. The biomedical monitoring system combining the mobile device of claim 3, wherein said mobile device comprises a modulation unit and said sensing device comprises a demodulation unit.

5. The biomedical monitoring system combining the mobile device of claim 2, wherein said microcontroller unit uses an encoding unit to encode said electrical datum and generate a digital signal; and after said processing unit receives said digital signal, said processing unit uses a decoding unit to decode said digital signal and give said electrical datum.

6. The biomedical monitoring system combining the mobile device of claim 5, wherein said mobile device comprises an encoding unit and said sensing device comprises a decoding unit.

7. The biomedical monitoring system combining the mobile device of claim 1, wherein said sensing device reads at least a kind of biomedical test strip.

8. The biomedical monitoring system combining the mobile device of claim 7, wherein said biomedical test strip is a blood-glucose test strip, a uric-acid test strip, or a cholesterol test strip.

9. The biomedical monitoring system combining the mobile device of claim 1, wherein said sensing device is a non-invasive blood glucose meter.

10. The biomedical monitoring system combining the mobile device of claim 1, wherein said mobile device comprises a memory unit connected electrically with said processing unit.

11. The biomedical monitoring system combining the mobile device of claim 10, wherein said memory unit stores an application program comprising an algorithm; and said processing unit uses said algorithm to convert said electrical datum to said biomedical datum.

12. The biomedical monitoring system combining the mobile device of claim 10, wherein said memory unit stores said biomedical datum.

13. The biomedical monitoring system combining the mobile device of claim 10, wherein said memory unit stores an application program; said application program editing said biomedical datum to a biomedical data file in the file format supported by said mobile device; and said memory unit stores said biomedical data file.

14. The biomedical monitoring system combining the mobile device of claim 1, wherein said mobile device comprises a displaying unit connected electrically with said processing unit and displaying said biomedical datum.

15. The biomedical monitoring system combining the mobile device of claim 1, wherein said displaying unit displays a graphic user interface.

16. The biomedical monitoring system combining the mobile device of claim 1, wherein said mobile device comprises an operating unit connected electrically with said processing unit and transmitting at least an operating signal to said processing unit.

17. The biomedical monitoring system combining the mobile device of claim 16, wherein said operating unit comprises a touch unit, a button unit, a gravity sensing unit, or a gyroscope unit; and said operating signal comprises a touch signal, a button signal, a movement signal, or a rotation signal.

18. The biomedical monitoring system combining the mobile device of claim 1, wherein said mobile device comprises an image extraction unit, connected electrically with said processing unit, extracting an image, and transmitting said image to said processing unit.

19. The biomedical monitoring system combining the mobile device of claim 18, wherein said image is given by said image extraction unit by extracting a barcode.

20. The biomedical monitoring system combining the mobile device of claim 1, wherein said mobile device comprises a voice unit connected electrically with said processing unit and outputting said biomedical datum in voice.

21. The biomedical monitoring system combining the mobile device of claim 1, wherein said mobile device comprises a terminal unit connected electrically with said processing unit and connected to the Internet.
